(19) United States
(12) Patent Application Publication
(10) Pub. No.: US 2024/0361297 A1
(43) Pub. Date: Oct. 31, 2024

(54) IDENTIFYING NUCLEOTIDES USING CHANGES IN IMPEDANCE BETWEEN ELECTRODES

(71) Applicant: Illumina, Inc., San Diego, CA (US)

(72) Inventor: Jeffrey Mandell, Rancho Santa Fe, CA (US)

(73) Assignee: Illumina, Inc., San Diego, CA (US)

(21) Appl. No.: 18/566,369

(22) PCT Filed: Oct. 24, 2022
(86) PCT No.: PCT/US2022/047600

§ 371 Date: Dec. 1, 2023

Related U.S. Application Data
(60) Provisional application No. 63/277,043, filed on Nov. 8, 2021.

(51) Int. Cl. G01N 33/487; C12Q 1/6869; G01N 27/327

(52) U.S. Cl. CPC G01N 33/48721; C12Q 1/6869; G01N 27/3278

(57) ABSTRACT

Identifying nucleotides using changes in impedance between electrodes is provided herein. In some examples. a method of identifying nucleotides includes sequentially passing, through a space between electrodes, labels respectively corresponding to the nucleotides. The labels may be used to sequentially alter an impedance between the electrodes. The sequential alterations in the impedance may be used to respectively identify the nucleotides. In some examples, a system for identifying nucleotides includes electrodes spaced apart from one another, labels corresponding to the nucleotides, and circuitry coupled to the electrodes. The labels may be passed through the space between the electrodes to alter an impedance between the electrodes. The circuitry may be configured to identify the nucleotides using the alterations in the impedance.

### CROSS-REFERENCE TO RELATED APPLICATION

This application claims the benefit of U.S. Provisional Patent Application No. 63/277,043, filed on Nov. 8, 2021 and entitled “IDENTIFYING NUCLEOTIDES USING CHANGES IN IMPEDANCE BETWEEN ELECTRODES”, the entire contents of which are incorporated by reference herein.

### BACKGROUND

A significant amount of academic and corporate time and energy has been invested into using nanopores to sequence polynucleotides. For example, the dwell time has been measured for complexes of DNA with the Klenow fragment (KF) of DNA polymerase I atop a nanopore in an applied electric field. Or, for example, a current or flux-measuring sensor has been used in experiments involving DNA captured in a α-hemolysin nanopore. Or, for example, KF-DNA complexes have been distinguished on the basis of their properties when captured in an electric field atop an α-hemolysin nanopore. In still another example, nucleic acid sequencing is performed using a single polymerase enzyme complex including a polymerase enzyme and a template nucleic acid attached proximal to a nanopore, and nucleotide analogs in solution. The nucleotide analogs include charge blockade labels that are attached to the polyphosphate portion of the nucleotide analog such that the charge blockade labels are cleaved when the nucleotide analog is incorporated into a growing nucleic acid. The charge blockade label is detected by the nanopore to determine the presence and identity of the incorporated nucleotide and thereby determine the sequence of a template nucleic acid. In still other examples, constructs include a transmembrane protein pore subunit and a nucleic acid handling enzyme.

However, such previously known compositions, systems, and methods may not necessarily be sufficiently robust, reproducible, or sensitive and may not have sufficiently high throughput for practical implementation, e.g., demanding commercial applications such as genome sequencing in clinical and other settings that demand cost effective and highly accurate operation. Accordingly, what is needed are improved compositions, systems, and methods for sequencing polynucleotides.

### SUMMARY

Identifying nucleotides using changes in impedance between electrodes is provided herein.

Some examples herein provide a method of identifying nucleotides. The method may include sequentially passing, through a space between electrodes, labels respectively corresponding to the nucleotides. The method may include sequentially altering an impedance between the electrodes using the labels. The method may include using the sequential alterations in the impedance to respectively identify the nucleotides.

In some examples, the electrodes are part of a resonating circuit that has a resonant frequency based upon the impedance between the electrodes. The sequential alterations in the impedance respectively may cause changes in the resonant frequency. The nucleotides may be identified using the respective changes in the resonant frequency. In some examples, the resonating circuit includes a tank circuit.

In some examples, the labels change the impedance by altering the capacitance within the resonating circuit that causes the changes in the resonant frequency.

In some examples, the sequential alterations in the impedance correspond to a sequence in which a polymerase adds the nucleotides to a first polynucleotide using a sequence of a second polynucleotide.

In some examples, the labels pass through the space between the electrodes while the labels are coupled to the nucleotides. In some examples, the labels pass through the space between the electrodes after the labels are cleaved from the nucleotides. In some examples, the labels pass through the space between the electrodes after the nucleotides are cleaved from a polynucleotide. In some examples, the labels are coupled to one another in a surrogate polymer passed through the space between the electrodes.

In some examples, the labels are coupled to respective gamma phosphates of the nucleotides.

In some examples, the space between the electrodes includes a nanopore. In some examples, the nanopore includes a solid state nanopore. In some examples, at least one of the electrodes includes a respective portion of the nanopore.

In some examples, at least one of the electrodes includes a surface of a fluidic channel. In some examples, each of the electrodes includes a respective surface of the fluidic channel.

In some examples, the electrodes are spaced apart from one another by about 100 nm or less.

In some examples, tips of the electrodes have widths of about 1 nm to about 100 nm.

In some examples, the labels pass through the space between the electrodes responsive to application of a voltage.

In some examples, the labels pass through the space between the electrodes at a rate of at least one thousand per second. In some examples, the labels pass through the space between the electrodes at a rate of at least ten thousand per second.

In some examples, the labels include polymers, beads, or particles having different physical characteristics than one another that alter the impedance between the electrodes. In some examples, the different physical characteristics are selected from the group consisting of permittivity, capacitance, inductance, magnetism, and ohmic resistance. In some examples, the particles are selected from the group consisting of metal particles, magnetic particles, and paramagnetic particles. In some examples, the beads are selected from the group consisting of polystyrene and polypropylene. In some examples, the polymers are selected from the group consisting of polyethylene glycol (PEG), protein, DNA, spermine, and spermidine.

Some examples herein provide a system for identifying nucleotides. The system may include electrodes spaced apart from one another. The system may include labels corresponding to the nucleotides, the labels being for passing through the space between the electrodes and for altering an impedance between the electrodes. The system may include circuitry coupled to the electrodes and configured to identify the nucleotides using the alterations in the impedance.

In some examples, the system includes a resonant circuit of which the electrodes are a part. The resonant circuit may have a resonant frequency based on the impedance between the electrodes. The sequential changes in the impedance caused by the labels respectively may cause changes in the resonant frequency. The nucleotides may be identified using the respective changes in the resonant frequency. In some examples, the resonating circuit includes a tank circuit.

In some examples, the labels change the impedance by altering the capacitance within the resonating circuit that causes the changes in the resonant frequency.

In some examples, the sequential alterations in the impedance correspond to a sequence in which a polymerase adds the nucleotides to a first polynucleotide using a sequence of a second polynucleotide.

In some examples, the labels pass through the space between the electrodes while the labels are coupled to the nucleotides. In some examples, the labels pass through the space between the electrodes after the labels are cleaved from the nucleotides. In some examples, the labels pass through the space between the electrodes after the nucleotides are cleaved from a polynucleotide. In some examples, the labels are coupled to one another in a surrogate polymer passed through the space between the electrodes. In some examples, the labels are coupled to respective gamma phosphates of the nucleotides.

In some examples, the space between the electrodes includes a nanopore. In some examples, the nanopore includes a solid state nanopore. In some examples, at least one of the electrodes includes a respective portion of the nanopore.

In some examples, at least one of the electrodes includes a surface of a fluidic channel. In some examples, each of the electrodes includes a respective surface of the fluidic channel.

In some examples, the electrodes are spaced apart from one another by about 100 nm or less.

In some examples, tips of the electrodes have widths of about 1 nm to about 100 nm.

In some examples, the labels pass through the space between the electrodes responsive to application of a voltage.

In some examples, the labels pass through the space between the electrodes at rate of at least one thousand per second. In some examples, the labels pass through the space between the electrodes at rate of at least ten thousand per second.

In some examples, the labels include polymers, beads or particles having different physical characteristics than one another that alter the impedance between the electrodes. In some examples, the different physical characteristics are selected from the group consisting of permittivity, capacitance, inductance, magnetism, and ohmic resistance. In some examples, the particles are selected from the group consisting of metal particles, magnetic particles, and paramagnetic particles. In some examples, the beads are selected from the group consisting of polystyrene and polypropylene. In some examples, the polymers are selected from the group consisting of polyethylene glycol (PEG), protein, DNA, spermine, and spermidine.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIGS. 1A-1C schematically illustrate an example composition and system for identifying nucleotides using changes in impedance between electrodes.

FIGS. 2A-2B schematically illustrate example signals that may be obtained using the system of FIGS. 1A-1C.

FIGS. 3A-3D schematically illustrate alternative example compositions and systems for identifying nucleotides using changes in impedance between electrodes.

FIGS. 4A-4B schematically illustrate alternative example compositions and systems for identifying nucleotides using changes in impedance between electrodes.

FIGS. 5A-5B schematically illustrate alternative example compositions and systems for identifying nucleotides using changes in impedance between electrodes.

FIGS. 6A-6B schematically illustrates example circuitry for use with the systems of FIGS. 1A-1C, 3A-3D, 4A-4B, or 5A-5B.

FIG. 7 illustrates a flow of operations in an example method for identifying nucleotides using changes in impedance between electrodes.

FIG. 8 illustrates an alternative configuration in which a biological pore resides in a membrane interposed between electrodes attached to the sides of a solid state aperture which serves to hold the membrane.

### DETAILED DESCRIPTION

Identifying nucleotides using changes in impedance between electrodes is provided herein.

For example, the present disclosure provides compositions, systems, and methods for polynucleotide sequencing that pass labels, corresponding to nucleotides, between electrodes. The labels may alter an impedance between the electrodes, and the change in impedance may be used to identify the nucleotides respectively corresponding to those labels. In some examples, the nucleotides may be used in a sequencing-by-synthesis (SBS) process. For example, a polymerase sequentially may add the nucleotides to a first polynucleotide based on the sequence of a second polynucleotide for which it is desired to determine the sequence. As the polymerase sequentially acts upon those nucleotides, the corresponding labels may pass between the electrodes and may induce measurable changes in impedance therebetween. The labels may be coupled to the nucleotides while passing between the electrodes, or may be cleaved from the nucleotides before passing between the electrodes. In other examples, the labels may be coupled to nucleotides that are cleaved from a polynucleotide, e.g., using a nuclease enzyme. In still other examples, the labels may be provided within surrogate polymers, the sequence of which corresponds to that of a polynucleotide being sequenced. In any such examples, labels having different impedances than one another respectively may be coupled to, or between, different nucleotides, and as such a given nucleotide may be identified based on the particular change to the impedance between the electrodes caused by the label to which that nucleotide is associated. As such, in various examples, the present compositions, systems, and methods are compatible with electrical-based identification of labeled nucleotides, relatively high-density flow cells, and as such may provide for relatively inexpensive sequencing instruments using relatively inexpensive consumables, with high sequencing throughput.

First, some terms used herein will be briefly explained. Then, some example compositions, example methods, and example systems including electrical detection circuitry that can be used for identifying nucleotides using changes in impedance between electrodes, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or system, the term “comprising” means that the compound, composition, or system includes at least the recited features or components, but may also include additional features or components.

As used herein, the singular forms “a”, “an” and “the” include plural referents unless the content clearly dictates otherwise.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar, backbone, and/or phosphate moiety compared to naturally occurring nucleotides. Nucleotide analogues also may be referred to as “modified nucleic acids.” Example modified nucleobases include inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates. Nucleotide analogues also include locked nucleic acids (LNA), peptide nucleic acids (PNA), and 5-hydroxylbutynl-2′-deoxyuridine (“super T”), and xNTPs.

As used herein “xNTP” refers to an expandable, 5′ triphosphate modified nucleotide substrate compatible with template dependent enzymatic polymerization. An xNTP includes two distinct functional components, namely, a nucleobase 5′-triphosphate and a tether or tether precursor that is attached within each nucleotide at positions that allow for controlled RT expansion by intra-nucleotide cleavage.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof such as locked nucleic acids (LNA) and peptide nucleic acids (PNA). A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA, LNA, or PNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

As used herein, a “polymerase” is intended to mean an enzyme having an active site that assembles polynucleotides by polymerizing nucleotides into polynucleotides. A polymerase can bind a primer and a single stranded target polynucleotide, and can sequentially add nucleotides to the growing primer to form a “complementary copy” polynucleotide having a sequence that is complementary to that of the target polynucleotide. DNA polymerases may bind to the target polynucleotide and then move down the target polynucleotide sequentially adding nucleotides to the free hydroxyl group at the 3′ end of a growing polynucleotide strand. DNA polymerases may synthesize complementary DNA molecules from DNA templates and RNA polymerases may synthesize RNA molecules from DNA templates (transcription). Polymerases may use a short RNA or DNA strand (primer), to begin strand growth. Some polymerases may displace the strand upstream of the site where they are adding bases to a chain. Such polymerases may be said to be strand displacing, meaning they have an activity that removes a complementary strand from a template strand being read by the polymerase.

Example polymerases include Bst DNA polymerase, 9° Nm DNA polymerase, Phi29 DNA polymerase, DNA polymerase I (E. coli), DNA polymerase I (Large), (Klenow) fragment, Klenow fragment (3′-5′ exo-), T4 DNA polymerase, T7 DNA polymerase, Deep VentR™ (exo-) DNA polymerase, Deep VentR™ DNA polymerase, DyNAzyme™ EXT DNA, DyNAzyme™ II Hot Start DNA Polymerase, Phusion™ High-Fidelity DNA Polymerase, Therminator™ DNA Polymerase, Therminator™ II DNA Polymerase, VentR® DNA Polymerase, VentR® (exo-) DNA Polymerase, RepliPHI™ Phi29 DNA Polymerase, rBst DNA Polymerase, rBst DNA Polymerase (Large), Fragment (IsoTherm™ DNA Polymerase), MasterAmp™ AmpliTherm™, DNA Polymerase, Taq DNA polymerase, Tth DNA polymerase, Tfl DNA polymerase, Tgo DNA polymerase, SP6 DNA polymerase, Tbr DNA polymerase, DNA polymerase Beta, and ThermoPhi DNA polymerase. In specific, nonlimiting examples, the polymerase is selected from a group consisting of Bst, Bsu, and Phi29. As the polymerase extends the hybridized strand, it can be beneficial to include single-stranded binding protein (SSB). SSB may stabilize the displaced (non-template) strand. Example polymerases having strand displacing activity include, without limitation, the large fragment of Bst (Bacillus stearothermophilus) polymerase, exo-Klenow polymerase or sequencing grade T7 exo-polymerase. Some polymerases degrade the strand in front of them, effectively replacing it with the growing chain behind (5′ exonuclease activity). Some polymerases have an activity that degrades the strand behind them (3′ exonuclease activity). Some useful polymerases have been modified, either by mutation or otherwise, to reduce or eliminate 3′ and/or 5′ exonuclease activity.

As used herein, the term “primer” is defined as a polynucleotide to which nucleotides may be added via a free 3′ OH group. A primer may include a 3′ block inhibiting polymerization until the block is removed. A primer may include a modification at the 5′ terminus to allow a coupling reaction or to couple the primer to another moiety. A primer may include one or more moieties, such as 8-oxo-G, which may be cleaved under suitable conditions, such as UV light, chemistry, enzyme, or the like. The primer length may be any suitable number of bases long and may include any suitable combination of natural and non-natural nucleotides. A target polynucleotide may include an “amplification adapter” or, more simply, an “adapter,” that hybridizes to (has a sequence that is complementary to) a primer, and may be amplified so as to generate a complementary copy polynucleotide by adding nucleotides to the free 3′ OH group of the primer.

As used herein, the term “plurality” is intended to mean a population of two or more different members. Pluralities may range in size from small, medium, large, to very large. The size of small plurality may range, for example, from a few members to tens of members. Medium sized pluralities may range, for example, from tens of members to about 100 members or hundreds of members. Large pluralities may range, for example, from about hundreds of members to about 1000 members, to thousands of members and up to tens of thousands of members. Very large pluralities may range, for example, from tens of thousands of members to about hundreds of thousands, a million, millions, tens of millions and up to or greater than hundreds of millions of members. Therefore, a plurality may range in size from two to well over one hundred million members as well as all sizes, as measured by the number of members, in between and greater than the above example ranges. Example polynucleotide pluralities include, for example, populations of about 1×10⁵ or more, 5×10⁵ or more, or 1×10⁶ or more different polynucleotides. Accordingly, the definition of the term is intended to include all integer values greater than two. An upper limit of a plurality may be set, for example, by the theoretical diversity of polynucleotide sequences in a sample.

As used herein, the term “double-stranded,” when used in reference to a polynucleotide, is intended to mean that all or substantially all of the nucleotides in the polynucleotide are hydrogen bonded to respective nucleotides in a complementary polynucleotide. A double-stranded polynucleotide also may be referred to as a “duplex.” As used herein, the term “single-stranded,” when used in reference to a polynucleotide, means that essentially none of the nucleotides in the polynucleotide are hydrogen bonded to a respective nucleotide in a complementary polynucleotide.

As used herein, the term “target polynucleotide” is intended to mean a polynucleotide that is the object of an analysis or action, and may also be referred to using terms such as “library polynucleotide,” “template polynucleotide,” or “library template.” The analysis or action includes subjecting the polynucleotide to amplification, sequencing and/or other procedure. A target polynucleotide may include nucleotide sequences additional to a target sequence to be analyzed. For example, a target polynucleotide may include one or more adapters, including an amplification adapter that functions as a primer binding site, that flank(s) a target polynucleotide sequence that is to be analyzed. In particular examples, target polynucleotides may have different sequences than one another but may have first and second adapters that are the same as one another. The two adapters that may flank a particular target polynucleotide sequence may have the same sequence as one another, or complementary sequences to one another, or the two adapters may have different sequences. Thus, species in a plurality of target polynucleotides may include regions of known sequence that flank regions of unknown sequence that are to be evaluated by, for example, sequencing (e.g., SBS). In some examples, target polynucleotides carry an amplification adapter at a single end, and such adapter may be located at either the 3′ end or the 5′ end the target polynucleotide. Target polynucleotides may be used without any adapter, in which case a primer binding sequence may come directly from a sequence found in the target polynucleotide.

The terms “polynucleotide” and “oligonucleotide” are used interchangeably herein. The different terms are not intended to denote any particular difference in size, sequence, or other property unless specifically indicated otherwise. For clarity of description, the terms may be used to distinguish one species of polynucleotide from another when describing a particular method or composition that includes several polynucleotide species.

As used herein, the term “substrate” refers to a material used as a support for compositions described herein. Example substrate materials may include glass, silica, plastic, quartz, metal, metal oxide, organo-silicate (e.g., polyhedral organic silsesquioxanes (POSS)), polyacrylates, tantalum oxide, complementary metal oxide semiconductor (CMOS), or combinations thereof. An example of POSS can be that described in Kehagias et al., Microelectronic Engineering 86 (2009), pp. 776-778, which is incorporated by reference in its entirety. In some examples, substrates used in the present application include silica-based substrates, such as glass, fused silica, or other silica-containing material. In some examples, silica-based substrates can include silicon, silicon dioxide, silicon nitride, or silicone hydride. In some examples, substrates used in the present application include plastic materials or components such as polyethylene, polystyrene, poly (vinyl chloride), polypropylene, nylons, polyesters, polycarbonates, and poly (methyl methacrylate). Example plastics materials include poly (methyl methacrylate), polystyrene, and cyclic olefin polymer substrates. In some examples, the substrate is or includes a silica-based material or plastic material or a combination thereof. In particular examples, the substrate has at least one surface including glass or a silicon-based polymer. In some examples, the substrates can include a metal. In some such examples, the metal is gold. In some examples, the substrate has at least one surface including a metal oxide. In one example, the surface includes a tantalum oxide or tin oxide. Acrylamides, enones, or acrylates may also be utilized as a substrate material or component. Other substrate materials can include, but are not limited to gallium arsenide, indium phosphide, aluminum, ceramics, polyimide, quartz, resins, polymers and copolymers. In some examples, the substrate and/or the substrate surface can be, or include, quartz. In some other examples, the substrate and/or the substrate surface can be, or include, semiconductor, such as GaAs or ITO. The foregoing lists are intended to be illustrative of, but not limiting to the present application. Substrates can include a single material or a plurality of different materials. Substrates can be composites or laminates. In some examples, the substrate includes an organo-silicate material.

Substrates can be flat, round, spherical, rod-shaped, or any other suitable shape. Substrates may be rigid or flexible. In some examples, a substrate is a bead or a flow cell.

Substrates can be non-patterned, textured, or patterned on one or more surfaces of the substrate. In some examples, the substrate is patterned. Such patterns may include posts, pads, wells, ridges, channels, or other three-dimensional concave or convex structures. Patterns may be regular or irregular across the surface of the substrate. Patterns can be formed, for example, by nanoimprint lithography or by use of metal pads that form features on non-metallic surfaces, for example.

In some examples, a substrate described herein forms at least part of a flow cell or is located in or coupled to a flow cell. Flow cells may include a flow chamber that is divided into a plurality of lanes or a plurality of sectors. Example flow cells and substrates for manufacture of flow cells that can be used in methods and compositions set forth herein include, but are not limited to, those commercially available from Illumina, Inc. (San Diego, CA).

As used herein, the term “electrode” is intended to mean a solid structure that conducts electrical current and which may be capable of propagating an electromagnetic field, including radio frequency (RF) signals of KHz, MHz, GHz, or THz frequencies. Electrodes may act as RF source electrodes and RF receiver electrodes, or both. Electrodes may act as RF source electrodes and counterpoise electrodes. Thus, one may appreciate that the electrodes may act interoperate to act as a reactively coupled pair with a space therebetween. Electrodes may include any suitable electrically conductive material, such as gold, palladium, or platinum, or combinations thereof. In some examples, an electrode may be disposed on a substrate, and pairs of electrodes may be coplanar. Electrode tips may be gapped. In some examples, an electrode may define a substrate. In some examples, electrodes may be positioned across a fluidically-conductive channel (the channel is coplanar with the electrodes, but at right angles to the electrodes), or across a fluidically-conductive pore that is oriented perpendicular to the electrode plane. In some examples, electrodes may be in direct contact with the fluid, for example, an electrolyte solution, or in some examples, may be insulated from the fluid. Electrodes may be shaped in various ways including, but not limited to, straight tips, tapered tips, and bowtie tips. Electrodes may be coupled to detection circuitry configured to detect a reflected RF signal or a transmitted RF signal. For example, the detection circuitry may be configured to measure a change in impedance between the electrodes responsive to passage of an element through the space between the electrodes. In various instances, electrodes may be spaced to establish a gap having a capacitive reactance, inductive reactance and/or resistance component which may change responsive to passage of the element through the space between the electrodes. Such a change in impedance (e.g., capacitive reactance, inductive reactance, and/or resistance) may cause detectable changes in a reflected RF signal or a transmitted RF signal. For instance, such a change in impedance may cause changes in resonant characteristics of a circuit including the electrodes. Such a change in impedance may cause changes in coupling of the RF signal across the electrodes. Such a change may result in a shift in the frequency of the reflected RF signal or transmitted RF signal, and the detection circuitry may detect such a frequency shift. Such a change may result in a shift in a ratio between transmitted power and reflected power across the gap between the electrodes (e.g., a standing wave ratio). The detection circuitry may measure one or more of these changes.

A “space between electrodes” refers to a gap or aperture between first and second electrodes. Electrodes having such a space between them may be referred to as gapped. In some examples, a fluid, such as a liquid or a gas, may pass through the space between the electrodes. Elements within the fluid, such as molecules, also may pass through the space between the electrodes. Responsive to detection circuitry applying an electric voltage bias between the electrodes, the space between the electrodes may include an electrical field. Moreover, responsive to detection circuitry applying a transient bias, pulse, sinusoidal signal (e.g., RF signal) across the electrodes, the space between the electrodes may include a varying electrical and/or magnetic field and, may establish the electrodes and space between the electrodes as an aspect of a circuit wherein energy is alternately stored in electrical and magnetic fields, exhibiting resonance, characteristics of which may be changed by a presence of elements with various dielectric properties in the space.

As used herein, the term “impedance” is intended to mean the effective resistance of an element to electrical current. The electrical current may be alternating. The element may include one or more components that may be part of an electrical circuit. For example, the element may include resistors, capacitors, and inductors. Any number of elements may be combined in a circuit in any combination in series and in parallel. Impedance may occur between a first and second electrodes disposed around any number of connected elements, and there may be a space between the first and second electrodes within which a fluid is disposed, and the impedance of such element may be based, in part, on the composition of the fluid. For example, the fluid may have a capacitive reactance arising from the effect of capacitance and/or an inductive reactance arising from the effect of inductance that contributes to the impedance of the element. Additionally, the fluid may have an ohmic resistance that contributes to the impedance of the element. Additionally, the respective interfaces between the fluid and the first and second electrodes may have ohmic resistances, inductive reactance, and capacitive reactance that respectively contribute to the impedance of the element. Illustratively, the element may have a different impedance when the fluid between the first and second electrodes includes a gas, as compared to when the fluid includes a liquid. As another example, the element may have a different impedance when the fluid includes a first label, as compared to when the fluid includes a second label that is different than the first label.

As used herein, a “tank circuit” or “LC circuit” is intended to mean an electrical circuit that includes an inductor (L) operatively coupled to a capacitor (C), and in which electrical energy oscillates between the inductor and the capacitor at a resonant frequency. The resonant frequency of the tank circuit is based, at least in part, on the inductance of the inductor and on the capacitance of the capacitor. A tank circuit may be operatively coupled to first and second electrodes that are spaced apart from one another, and together with a material (such as a fluid) within the space between the electrodes may form an element of the tank circuit with an impedance. Any elements within such a fluid may alter the impedance of the tank circuit by contributing additional capacitance and/or inductance to the circuit. As such, an element may become operatively coupled to the tank circuit, and in so doing, cause respective changes in the resonant frequency of the tank circuit that may be detected by an appropriate detection circuit. For nonlimiting examples of tank circuits, nanopores, electrodes, and detection circuits, see the following references, the entire contents of each of which are incorporated by reference herein: Bhat et al., “A tank-circuit for ultrafast single particle detection in micropores,” Phys. Rev. Lett. 121 (7): 078102, 1-5 (2018); Bhat et al., “Radio Frequency Tank Circuit for Probing Planar Lipid Bilayers,” Soft Nanoscience Letters 3 (4): 87-92 (2013); Kim et al., “Radio-frequency response of single pores and artificial ion channels,” New Journal of Physics 13:093033 (2011); Ramachandran et al., “Direct microwave transmission on single α-hemolysin pores,” Applied Physics Letters 99 (9): 093105 (2011); Schoelkopf et al., “The radio-frequency single-electron transistor (RF-SET): A fast and ultra-sensitive electrometer,” Science 280 (5367), 1238-1242 (1998); and U.S. Patent Publication No. 2019/0293623 to Blick et al.

As used herein, the term “pore” is intended to mean a structure that includes an aperture that permits molecules to cross therethrough from a first side of the pore to a second side of the pore. That is, the aperture extends through the first and second sides of the pore. Molecules that can cross through an aperture of a pore can include, for example, ions or water-soluble molecules such as amino acids or nucleotides. The pore can be disposed within a barrier, or can be provided through a substrate. When at least a portion of the aperture of a pore has a width of 100 nm or less, e.g., 10 nm or less, or 2 nm or less, the pore can be, but need not necessarily be, referred to as a “nanopore.” Optionally, a portion of the aperture can be narrower than one or both of the first and second sides of the pore, in which case that portion of the aperture can be referred to as a “constriction.” Alternatively or additionally, the aperture of a pore, or the constriction of a pore (if present), or both, can be greater than 0.1 nm, 0.5 nm, 1 nm, 10 nm or more. A pore can include multiple constrictions, e.g., at least two, or three, or four, or five, or more than five constrictions. Pores include biological pores, solid state pores, or biological and solid state hybrid pores.

Biological pores include, for example, polypeptide pores and polynucleotide pores. A “polypeptide pore” is intended to mean a pore that is made from one or more polypeptides. The one or more polypeptides can include a monomer, a homopolymer or a heteropolymer. Structures of polypeptide pores include, for example, an α-helix bundle pore and a β-barrel pore as well as all others well known in the art. Example polypeptide pores include α-hemolysin, Mycobacterium smegmatis porin A, gramicidin A, maltoporin, OmpF, OmpC, PhoE, Tsx, F-pilus, SP1, mitochondrial porin (VDAC), Tom40, outer membrane phospholipase A, and Neisseria autotransporter lipoprotein (NaIP). “Mycobacterium smegmatis porin A (MspA)” is a membrane porin produced by Mycobacteria, allowing hydrophilic molecules to enter the bacterium. MspA forms a tightly interconnected octamer and transmembrane beta-barrel that resembles a goblet and includes a central constriction. For further details regarding α-hemolysin, see U.S. Pat. No. 6,015,714, the entire contents of which are incorporated by reference herein. For further details regarding SP1, see Wang et al., Chem. Commun., 49:1741-1743 (2013), the entire contents of which are incorporated by reference herein. For further details regarding MspA, see Butler et al., “Single-molecule DNA detection with an engineered MspA protein nanopore,” Proc. Natl. Acad. Sci. 105:20647-20652 (2008) and Derrington et al., “Nanopore DNA sequencing with MspA,” Proc. Natl. Acad. Sci. USA, 107:16060-16065 (2010), the entire contents of both of which are incorporated by reference herein. Other pores include, for example, the MspA homolog from Norcadia farcinica, and lysenin. For further details regarding lysenin, see PCT Publication No. WO 2013/153359, the entire contents of which are incorporated by reference herein.

A “polynucleotide pore” is intended to mean a pore that is made from one or more nucleic acid polymers. A polynucleotide pore can include, for example, a polynucleotide origami.

A “solid state pore” is intended to mean a pore that is made from one or more materials that are not of biological origin. A solid-state pore can be made of inorganic or organic materials. Solid state pores include, for example, silicon nitride pores, silicon dioxide pores, and graphene pores. A solid state pore may include one or more electrodes. For example, a solid state pore may include first and second electrodes, and a space between the electrodes may include an aperture of the pore. The first and second electrodes may be disposed on a substrate, and the aperture of the pore may extend through the substrate such that a fluid may pass through the substrate via the aperture.

A “biological and solid state hybrid pore” is intended to mean a hybrid pore that is made from materials of both biological and non-biological origins. Materials of biological origin are defined above and include, for example, polypeptides and polynucleotides. A biological and solid state hybrid pore includes, for example, a polypeptide-solid state hybrid pore and a polynucleotide-solid state pore. In one nonlimiting example, a solid-state pore holds a biological pore. The resulting biological and solid state hybrid pore may be located between two electrodes operatively coupled to a detection circuit, e.g., tank circuit. Elements (e.g., labels) passing through the biological pore may be expected to change the impedance of the hybrid pore, which may be detected by the tank circuit. See, e.g., Ramachandran et al., “Direct microwave transmission on single α-hemolysin pores,” Applied Physics Letters 99(9): 093105 (2011). Moreover, elements (e.g., labels) passing through the biological pore may be expected to change a ratio of electromagnetic power (i) coupled across the pore through the electrodes and (ii) reflected by the pore and associated structures.

As used herein, a “barrier” is intended to mean a structure that normally inhibits passage of molecules from one side of the barrier to the other side of the barrier. The molecules for which passage is inhibited can include, for example, ions or water soluble molecules such as nucleotides and amino acids. However, if a pore is disposed within a barrier, then the aperture of the pore may permit passage of molecules from one side of the barrier to the other side of the barrier. As one specific examples, if an aperture (such as a nanopore) is disposed within a barrier, the aperture of the nanopore may permit passage of molecules from one side of the barrier to the other side of the barrier. Barriers include membranes of biological origin, such as lipid bilayers, and non-biological barriers such as solid state membranes or substrates.

As used herein, “of biological origin” refers to material derived from or isolated from a biological environment such as an organism or cell, or a synthetically manufactured version of a biologically available structure.

As used herein, “solid state” refers to material that is not of biological origin.

As used herein, “linker” is intended to mean an elongated member having a head region, a tail region, and an elongated body therebetween. A linker can include a molecule. A linker can be, but need not necessarily be, in an elongated state, e.g., can include an elongated molecule. For example, an elongated body of a linker can have secondary or tertiary configurations such as hairpins, folds, helical configurations, or the like. Linkers can include polymers such as polynucleotides or synthetic polymers. Linkers can have lengths (e.g., measured in a stretched or maximally extended state) ranging, for example, from about 5 nm to about 500 nm, e.g., from about 10 nm to about 100 nm. Linkers can have widths ranging, for example, from about 1 nm to about 50 nm, e.g., from about 2 nm to about 20 nm. Linkers can be linear or branched. As used herein, a “head region” of a linker is intended to mean a functional group at one end of the linker that is attached to another member, and a “tail region” of a linker is intended to mean a functional group at the other end of the linker that is attached to another member. Such attachments of the head region and tail region respectively can be formed via a chemical bond, e.g., via a covalent bond, hydrogen bond, ionic bond, dipole-dipole bond, London dispersion forces, or any suitable combination thereof. In one example, such attachment can be formed through hybridization of a first oligonucleotide of the head region to a second oligonucleotide of another member. Alternatively, such attachment can be formed using physical or biological interactions, e.g., an interaction between a first protein structure of the head region and a second protein structure of the other member that inhibits detachment of the head region from the other member. Example members to which a head region or a tail region of a linker can be attached include a label and a molecule, such as a nucleotide.

As used herein, an “elongated body” is intended to mean a portion of a member, such as a linker, that extends between the head region and the tail region. An elongated body can be formed of any suitable material of biological origin or nonbiological origin, or a combination thereof. In one example, the elongated body includes a polymer. Polymers can be biological or synthetic polymers. Example biological polymers that suitably can be included within an elongated body include polynucleotides, polypeptides, polysaccharides, polynucleotide analogs, and polypeptide analogs. Example polynucleotides and polynucleotide analogs suitable for use in an elongated body include DNA, enantiomeric DNA, RNA, PNA (peptide-nucleic acid), morpholinos, and LNA (locked nucleic acid). Example synthetic polypeptides can include charged amino acids as well as hydrophilic and neutral residues. Example synthetic polymers that suitably can be included within an elongated body include PEG (polyethylene glycol), PPG (polypropylene glycol), PVA (polyvinyl alcohol), PE (polyethylene), LDPE (low density polyethylene), HDPE (high density polyethylene), polypropylene, PVC (polyvinyl chloride), PS (polystyrene), NYLON (aliphatic polyamides), TEFLON™ (tetrafluoroethylene), thermoplastic polyurethanes, polyaldehydes, polyolefins, poly (ethylene oxides), poly (w-alkenoic acid esters), poly (alkyl methacrylates), and other polymeric chemical and biological linkers such as described in Hermanson, Bioconjugate Techniques, third edition, Academic Press, London (2013), the entire contents of which are incorporated by reference herein.

As used herein, the term “label” is intended to mean an element that passes between electrodes in such a manner as to cause a change in the electrical characteristics of a circuit including the electrodes, such as impedance or conductivity, and based upon which change a nucleotide corresponding to that label may be identified. In examples provided herein, labels can correspond to nucleotides by being attached to such nucleotides, by being attached and subsequently detached from such nucleotides, by being coupled to one another between nucleotides in a surrogate polymer, or by otherwise being associated with nucleotides.

As used herein, a “surrogate polymer” is intended to mean an elongated chain of labels having a sequence corresponding to a sequence of nucleotides in a polynucleotide. An XPANDOMER™ is a particular type of surrogate polymer developed by Roche Sequencing, Inc. (Pleasanton, CA). XPANDOMERS™ may be prepared using Sequencing By expansion™ (SBX™, Roche Sequencing, Pleasanton CA). In Sequencing by expansion™, an engineered polymerase polymerizes xNTPs which include nucleobases coupled to labels via linkers, using the sequence of a target polynucleotide. The polymerized nucleotides are then processed to generate an elongated chain of the labels, separated from one another by linkers which are coupled between the nucleotides, and having a sequence that is complementary to that of the target polynucleotide. For example descriptions of XPANDOMERS™, linkers (tethers), labels, engineered polymerases, and methods for SBX™, see the following patents, the entire contents of each of which are incorporated by reference herein: U.S. Pat. Nos. 7,939,249, 8,324,360, 8,349,565, 8,586,301, 8,592,182, 9,670,526, 9,771,614, 9,920,386, 10,301,345, 10,457,979, 10,676,782, 10,745,685, 10,774,105, and 10,851,405.

Identifying Nucleotides Using Changes in Impedance between Electrodes

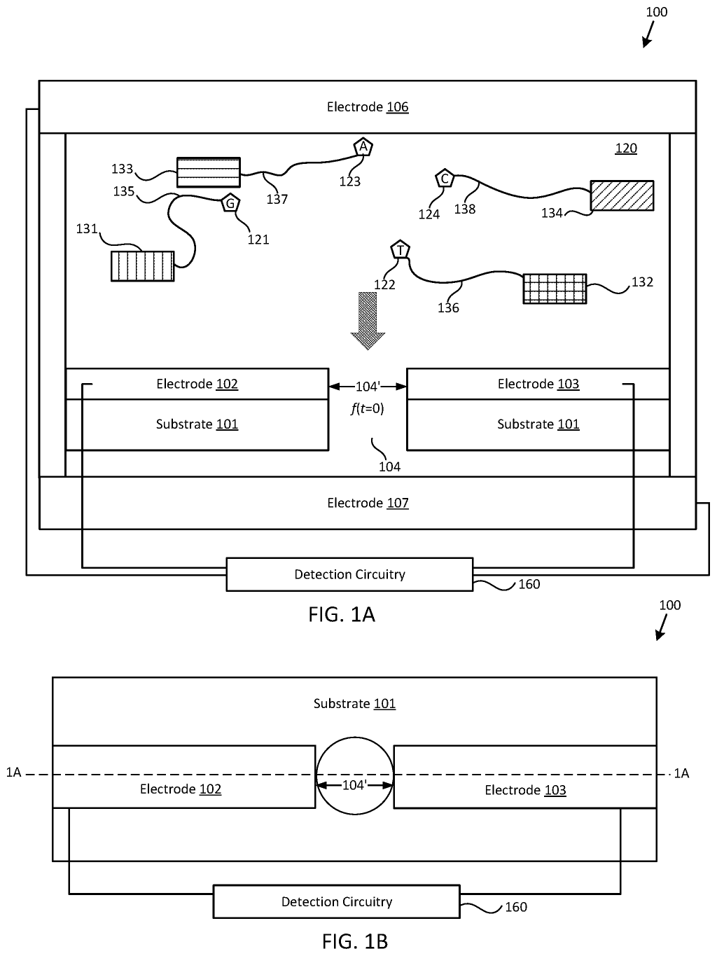

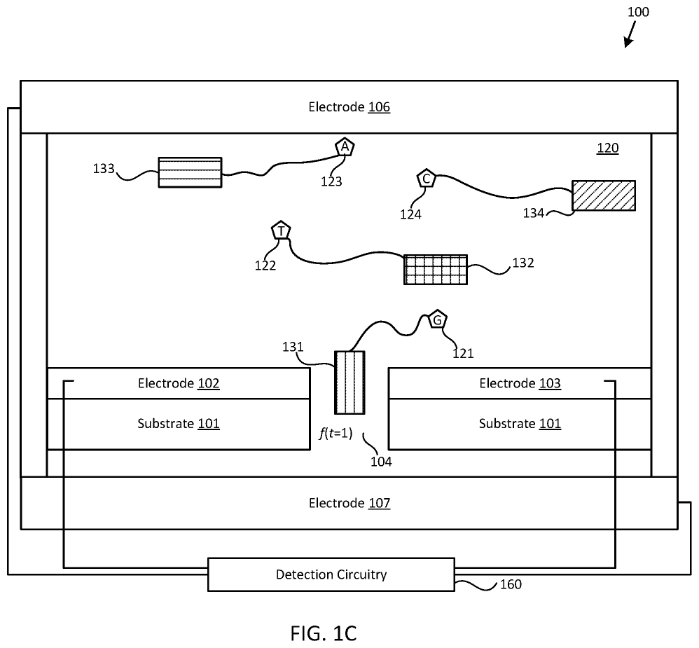

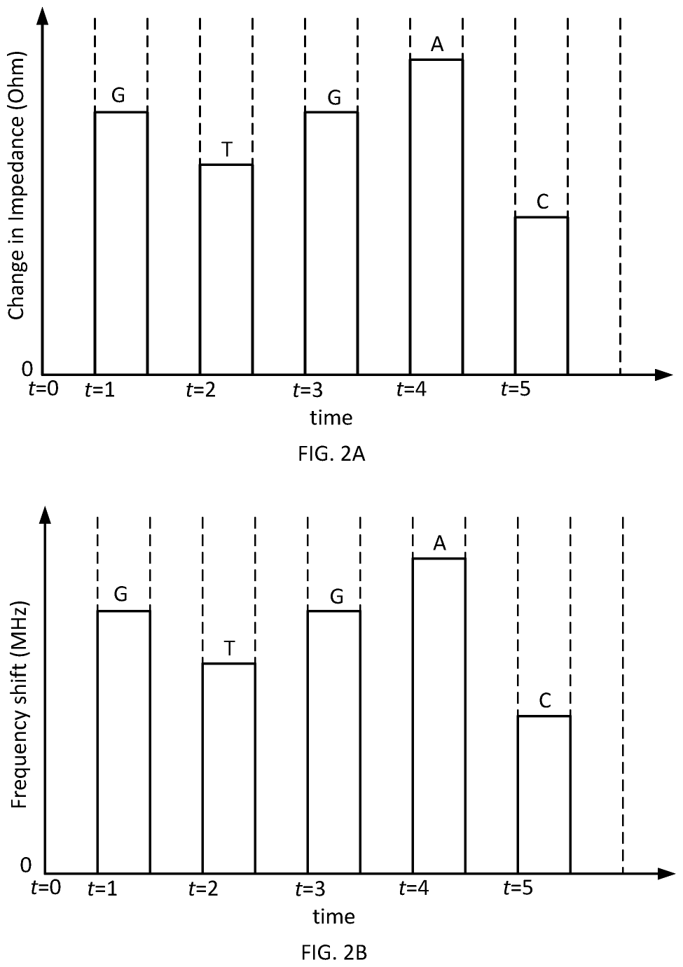

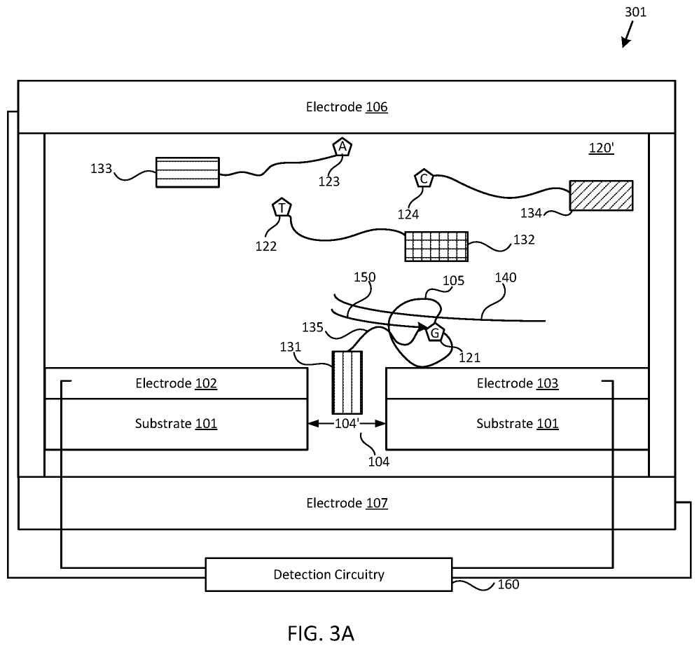

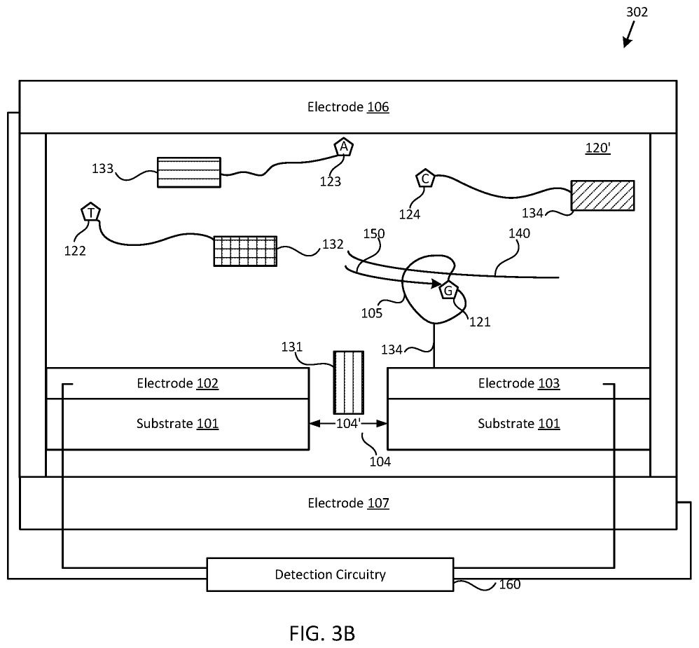

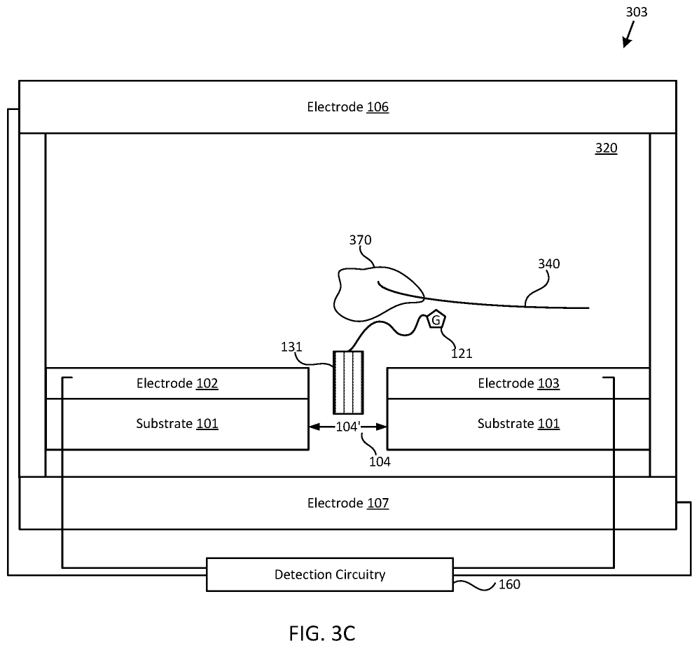

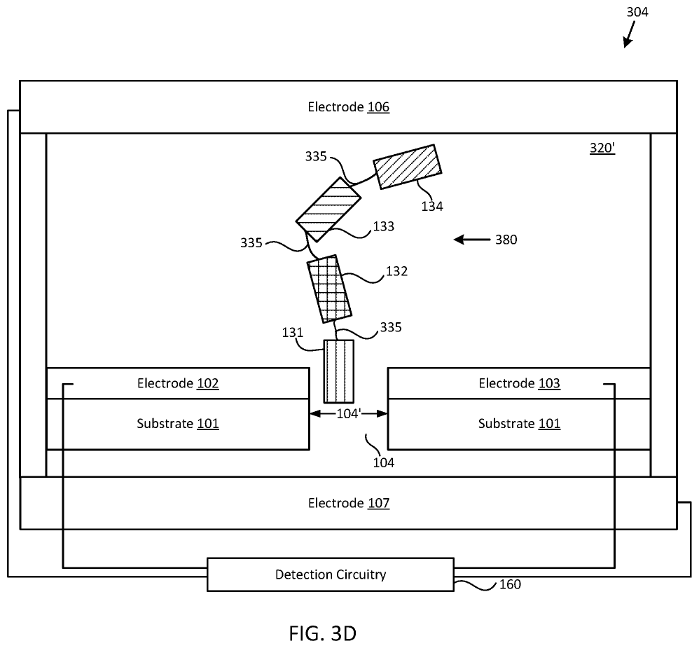

Some example compositions, systems, and methods for identifying nucleotides using changes in impedance between electrodes now will be described with reference to FIGS. 1A-1C and 2A-2B. Other nonlimiting examples will be described with reference to FIGS. 3A-3C, 4A-4B, 5A-5C, 6A-6B, 7, and 8.

FIGS. 1A-1C schematically illustrate an example composition and system for identifying nucleotides using changes in impedance between electrodes. System 100 illustrated in cross-section in FIG. 1A and in simplified plan view in FIG. 1B includes first electrode 102; second electrode 103 spaced apart from first electrode 102; aperture 104; third electrode 106; fourth electrode 107; fluid 120 including labels 131, 132, 133, and 134; and detection circuitry 160 coupled to electrodes 102, 103, 106, and 107 and configured to identify nucleotides 121, 122, 123, 124 to which labels 131, 132, 133, and 134 respectively correspond using alterations in impedance caused by the labels.

In the nonlimiting example illustrated in FIGS. 1A-1B, first electrode 102 and second electrode 103 may be disposed upon substrate 101, third electrode 106 may be spaced apart from aperture 104 on a first side of the aperture, and fourth electrode 107 may be spaced apart from aperture 104 on a second side of the aperture. Electrodes 102 and 103 may be used to detect labels 131, 132, 133, and 134 as such labels pass through aperture 104, e.g., in a manner such as described in greater detail herein. Detection circuitry 160 may be configured to apply a voltage bias across electrodes 106 and 107 to cause translocation of elements, such as labels 131, 132, 133, and 134, through aperture 104. Other nonlimiting structures of electrodes 102, 103, 106, and 107 are provided elsewhere herein.

Substrate 101 may have any suitable structure that normally inhibits current flow across the substrate, and that permits labels 131, 132, 133, 134 to pass through a space between electrode 102, 103. In the nonlimiting example illustrated in FIGS. 1A-1B, an aperture 104 is provided through substrate 101, through which aperture fluid 120 may flow from one side of the substrate to the other side of the substrate. Electrodes 102, 103 are spaced apart from one another by space 104′, such that fluid 120 flowing through aperture 104 also flows through space 104′. Space 104′ may have substantially the same dimension in

FIG. 1A as does aperture 104. For example, the distance between the points at which electrodes 102, 103 are closest to one another may be approximately the same as the points at which the sides of aperture 104 are farthest from one another. Illustratively, space 104′ may be formed in a common process as aperture 104. For example, electrodes 102, 103 may be disposed on substrate 101, and then space 104′ and aperture 104 may be concurrently formed using any suitable process, such as laser ablation (illustratively, using an excimer laser), drilling, photolithography, focused ion beam (FIB), or the like. Alternatively, substrate 101 including aperture 104 may be prepared, and then electrodes 102, 103 may be disposed on substrate 101.

An array of apertures 104 on a common substrate, or on any suitable number of substrates, may be prepared. In some examples the apertures of the array optionally share common fluidic chambers above and below the apertures, with each aperture having its own set of electrodes 102, 103, 106, and 107. Additionally, or alternatively, the array of apertures may share common fluidic chambers above and below the apertures, and may share common electrodes 106 and 107, with each aperture having its own set of electrodes 102 and 103. In either example, the fluidic chamber on the first side of the aperture (the same side as electrode 106) may include a library of polynucleotides, such as DNA, to sequence.

It will be appreciated that FIGS. 1A-1C illustrate only one example of an electrode structure that may be used to detect changes in impedance caused by labels corresponding to nucleotides. In the nonlimiting examples illustrated in FIGS. 1A-1C and 3A-3D, first and second electrodes 102, 103 may be considered to include a portion of a pore. Substrate 101, if provided, also may be considered to include a portion of the pore. The pore provided by electrodes 102, 103, and optional substrate 101 may be or include a nanopore, e.g., a solid state nanopore or a biological and solid state hybrid nanopore. Other nonlimiting structures for electrodes that are spaced apart from one another are provided elsewhere herein.

In the nonlimiting example illustrated in FIG. 1A, fluid 120 may include a first nucleotide analogue including first nucleotide 121 (e.g., G) coupled to first label 131 via linker 135; a second nucleotide analogue including second nucleotide 122 (e.g., T) coupled to second label 132 via linker 136; a third nucleotide analogue including third nucleotide 123 (e.g., A) coupled to third label 133 via linker 137; and a fourth nucleotide analogue including fourth nucleotide 124 (e.g., C) coupled to fourth label 134 via linker 138. Detection circuitry 160, together with first and second electrodes 102, 103, may include a tank circuit having a resonant frequency that is based, in part, on the ohmic resistance of fluid 120 and any molecules that may pass between space 104′ at a given time. At the particular time illustrated in FIG. 1A, neither any of the nucleotides nor any of the labels or linkers is passing through space 104′, and as such the resonant frequency may have a first value, as indicated by “f(t=0)” in FIG. 1A.

The labels 131, 132, 133, 134 of the nucleotide analogues may pass between electrodes 102, 103 and alter an impedance between such electrodes such that detection circuitry 160 respectively may identify nucleotides 121, 122, 123, 124. For example, labels 131, 132, 133, 134 may have different physical characteristics than one another. Stated broadly, labels 131, 132, 133, 134 may have different electrical and magnetic properties. Labels 131, 132, 133, 134 may exhibit capacitance and/or inductance. Labels 131, 132, 133, and 134 may have different dielectric characteristics, such as permittivity, capacitance, inductance, magnetism, and ohmic resistance. Such physical characteristics may differently alter the capacitive portion of the impedance between the electrodes, thus differently affecting the resonant frequency of the circuit, from which the identity of the corresponding nucleotide 121, 122, 123, 124 may be identified, e.g., in a manner such as will now be described with reference to FIG. 1C and FIGS. 2A-2B. For example, at the particular time illustrated in FIG. 1C, first label 131, which corresponds to (and in this example is coupled to) first nucleotide 121, may pass through space 104′. First label 131 within space 104′ may have a first permittivity that detectably alters the impedance between electrodes 102, 103 in such a manner that detection circuit 160 may identify first nucleotide 121 as corresponding to that label. For example, first label 131 may measurably change the resonant frequency of the tank circuit including electrodes 102, 103 and detection circuit 160 to a second value, as indicated by “f(t=1)” in FIG. 1C and nucleotide 121 may be identified using such change in the resonant frequency. At other times not specifically illustrated, labels 132, 133, and 134 may pass through space 104′ and may respectively detectably alter the impedance between electrodes 102, 103 in such a manner that detection circuit 160 may identify the nucleotides 122, 123, 124 as respectively corresponding to those labels.

For example, because the respective labels 131, 132, 133, and/or 134 may have different permittivities than one another, such labels may cause different impedances between electrodes 102, 103 than one another based upon which the corresponding nucleotides 121, 122, 123, 124 respectively may be identified. For example, FIGS. 2A-2B schematically illustrates example signals that may be obtained using the system of FIGS. 1A-1C. It will be appreciated that the particular sequence and timing with which labels 131, 132, 133, 134 pass through space 104′ between electrodes 102, 103 is for illustrative purposes, and that although FIGS. 2A-2B may appear to suggest that the passage of the labels through the space happens at regular intervals, the actual timing of passage may vary significantly. Moreover, while, for ease of illustration, various magnitudes of impedance and frequency shift and various relationships between impedance and frequency shift are shown in FIGS. 2A-2B, one may appreciate that these magnitudes and relationships are illustrative and may be different for different tank circuit architectures. In some examples the labels may pass between the electrodes substantially one at a time, thus facilitating distinguishing between the changes in impedance caused by such labels.

At an initial time (t=0, such as illustrated in FIG. 1A), fluid 120 passes through space 104′ and substantially no labels pass through such space, and accordingly detection circuitry 160 measures a baseline impedance between electrodes 102, 103 in a manner such as illustrated in FIG. 2A. At a subsequent time (+=1, such as illustrated in FIG. 1C), label 131 passing through space 104′ alters the impedance between electrodes 102, 103, resulting in a change in the first impedance value corresponding to nucleotide 121 (e.g., G). In some examples, the capacitive change to the impedance caused by label 131 may be in the range of fF (femto-Farad) to aF (atto-Farad) to zF (zepto-Farad), for example. For example, it has been calculated that the capacitance of DNA nucleotides may be in the range of about 10-21 F (zF), and that the effective dielectric constant for DNA nucleotides is approximately between 2 and 5; see Lu et al., “Nucleotide capacitance calculation for DNA sequencing,” Biophysical Journal: Biophysical Letters 95 (9): L60-L62 (2008), the entire contents of which are incorporated by reference herein. Additionally, beta-cyclodextrin transiting an α-hemolysin nanopore was measured to cause an approximately 50 aF capacitive change; see Kim et al., “Radio-frequency response of single pores and artificial ion channels,” New Journal of Physics 13:093033 (2011). The present labeled nucleotides (e.g., surrogate polymers) may be configured to cause significantly greater changes in impedance than unlabeled DNA nucleotides (as in Lu et al.) or than beta-cyclodextrin (as in Kim et al.), and thus readily may be characterized, e.g., based on frequency changes caused by such changes in impedance.

After label 131 passes through space 104′, the impedance may return to baseline (at which the change in impedance is zero). At a subsequent time (t=2), label 132 passing through space 104′ alters the impedance between electrodes 102, 103, resulting in a second change in the impedance value corresponding to nucleotide 122 (e.g., T). After label 132 passes through space 104′, the impedance may return to baseline. At a subsequent time (t=3), another label 131 passing through space 104′ alters the impedance between electrodes 102, 103, resulting in the first change in impedance value corresponding to another nucleotide 121 (e.g., G). Note that the first changes in impedance values measured at t=1 and t=3 are about the same as one another, because both of nucleotides 121 were coupled to a respective label 131 causing the same change in impedance. After label 131 passes through space 104′, the impedance may return to baseline. At a subsequent time (t=4), label 133 passing through space 104′ alters the impedance between electrodes 102, 103, resulting in a third impedance value corresponding to nucleotide 123 (e.g., A). After label 133 passes through space 104′, the impedance may return to baseline. At a subsequent time (t=5), label 134 passing through space 104′ alters the impedance between electrodes 102, 103, resulting in a fourth change in impedance value corresponding to nucleotide 124 (e.g., C). After label 134 passes through space 104′, the impedance may return to baseline. Thus, it may be understood that detection circuitry 160 may be configured to measure a baseline impedance between labels, and may be configured to measure changes in impedance with values that may correspond to the particular labels to which nucleotides correspond. As such, the nucleotides may be identified using such changes in impedances.

In examples in which a tank circuit includes detection circuitry 160 and electrodes 102, 103, the tank circuit may have a resonant frequency that labels 131, 132, 133, 134 alter in a manner such as illustrated in FIG. 2B. At an initial time (t=0, such as illustrated in FIG. 1A), fluid 120 passes through space 104′ and substantially no labels pass through such space, and accordingly the tank circuit has a baseline resonance frequency, corresponding to a frequency shift of zero as illustrated in FIG. 2B. At a subsequent time (t=1, such as illustrated in FIG. 1C), label 131 passing through space 104′ alters the impedance between electrodes 102, 103, resulting in a change in the resonant characteristics of the tank circuit, which in various examples causes a first frequency shift corresponding to nucleotide 121 (e.g., G). After label 131 passes through space 104′, the frequency shift may return to zero. At a subsequent time (t=2), label 132 passing through space 104′ alters the impedance between electrodes 102, 103, resulting in a second frequency shift corresponding to nucleotide 122 (e.g., T). After label 132 passes through space 104′, the frequency shift may return to zero. At a subsequent time (t=3), another label 131 passing through space 104′ alters the impedance between electrodes 102, 103, resulting in the first frequency shift corresponding to another nucleotide 121 (e.g., G). Note that the first frequency shifts measured at t=1 and t=3 are about the same as one another, because both of nucleotides 121 were coupled to a respective label 131 causing the same change in impedance. After label 131 passes through space 104′, the frequency shift may return to zero. At a subsequent time (t=4), label 133 passing through space 104′ alters the impedance between electrodes 102, 103, resulting in a third frequency shift corresponding to nucleotide 123 (e.g., A). After label 133 passes through space 104′, the frequency shift may return to zero. At a subsequent time (t=5), label 134 passing through space 104′ alters the impedance between electrodes 102, 103, resulting in a fourth frequency shift corresponding to nucleotide 124 (e.g., C). After label 134 passes through space 104′, the frequency shift may return to zero. Thus, it may be understood that detection circuitry 160 may be configured to measure a baseline frequency corresponding to the presence of fluid 120 (without labels) between electrodes 102, 103, and may be configured to measure frequency shifts with values that may correspond to the particular labels to which nucleotides correspond. As such, the nucleotides may be identified using such frequency shifts.

It will be appreciated that the present systems and methods suitably may be adapted to identify nucleotides in any suitable context or application, and that the labels need not necessarily be coupled to the nucleotides in order to correspond to the nucleotides. For example, FIGS. 3A-3D schematically illustrate alternative example compositions and systems for identifying nucleotides using changes in impedance between electrodes. FIG. 3A illustrates system 301 which is configured similarly as system 100 and configured to identify nucleotide 121 during a sequencing-by-synthesis (SBS) process in which label 131 is coupled to nucleotide 121. More specifically, modified fluid 120′ may be configured similarly as fluid 120 and also may include polymerase 105, first polynucleotide 140, and second polynucleotide 150. Alternatively, polymerase 105 may be coupled to substrate 101 or to either of electrodes 102, 103 in any suitable manner, such as via a linker (not specifically illustrated). As illustrated in FIG. 3A, polymerase 105 may add nucleotide 121 (or any other nucleotide 122, 123, 124 within fluid 120) to second polynucleotide 150 based on a sequence of first polynucleotide 140. Illustratively, the next base in first polynucleotide 140 may be C, based upon which polymerase 105 may add first nucleotide 121 (G) to second polynucleotide 150. First label 131, which in the illustrated example is coupled to first nucleotide 121, may alter the impedance between electrodes 102, 103 based upon which detection circuitry 160 may identify nucleotide 121. Label 131 (in addition to labels 132, 133, and 134) may be detachable, by polymerase 105, from the respective nucleotide so as to be transportable away from space 104′ so as to return to a baseline impedance corresponding to presence of fluid 120 within the space (e.g., a baseline frequency shift of zero for the tank circuit, if one is used). For example, after adding nucleotide 121 to second polynucleotide 150, polymerase 105 may cleave linker 135 so as to detach label 131 from nucleotide 121.

After polymerase 105 cleaves linker 135 from nucleotide 121, the polymerase may add another nucleotide to second polynucleotide 150 based on a sequence of first polynucleotide 140. Illustratively, the next base in first polynucleotide 140 may be A, based upon which polymerase 105 may add second nucleotide 122 (T) to second polynucleotide 150 in a manner such as illustrated in FIG. 3A. Second label 132, which is coupled to second nucleotide 122, may induce a second change in impedance in a manner similar to that described with reference to FIG. 2A. For example, label 131 may cause a second frequency shift in a manner such as described with reference to FIG. 2B. Polymerase 105 may add additional nucleotides to second polynucleotide 150 in a similar manner as described for nucleotides 121 and 122, and the labels coupled to such nucleotides may cause additional changes in impedance which are measured by detection circuitry 160.

Note that although FIGS. 1A-1C and 3A illustrate labels 131, 132, 133, 134 being coupled to respective nucleotides 121, 122, 123, 124 at the time such labels pass between electrodes 102, 103 and are detected, the labels instead may be decoupled from the nucleotides before such labels pass between the electrodes. For example, FIG. 3B illustrates system 302 which is configured similarly as system 301, and in which polymerase 105 is coupled to substrate 101 or to either of electrodes 102, 103 via linker 134. Linker 134 may be configured so as to sufficiently space polymerase 105 apart from space 104′ that labels 131, 132, 133, 134 may pass through the space after being cleaved from their corresponding nucleotides. In one nonlimiting example, linker 134 may be or include a membrane spanning protein (MSP).

Although FIGS. 3A-3B illustrate use of the present systems and methods for SBS, other implementations may be envisioned, some of which may relate to sequencing a polynucleotide. For example, FIG. 3C illustrates system 303 which may be configured similarly as system 100 and including alternative fluid 320 which includes polynucleotide 340 and nuclease 370 configured to individually cleave nucleotides from the polynucleotide. One, some, or all of the nucleotides within polynucleotide 340 respectively may be coupled to labels. As the nuclease 370 cleaves the nucleotides from polynucleotide 340, those nucleotides and the associated labels may pass through aperture 104 and be interrogated and identified. For example, at the particular time illustrated in FIG. 3C, nuclease 370 has cleaved nucleotide 121 from polynucleotide 340, and after such cleavage label 131 passes through space 104′ for detection in a manner similar to that described elsewhere herein. By repeating use of nuclease 370 to cleave nucleotides from polynucleotide 340 and detecting of the corresponding labels, polynucleotide 340 may be sequenced.

As another example, FIG. 3D illustrates system 304 which may be configured similarly as system 100 and including alternative fluid 320′ which includes surrogate polymer 380 passing through space 104′ between electrodes 102, 103. Surrogate polymer 380 may include a sequence of labels 131, 132, 133, 134 that are coupled together (e.g., via linkers 335) in a sequence corresponding to the sequence of nucleotides 121, 122, 123, 124 within a target polynucleotide (not specifically illustrated). As labels 131, 132, 133, 134 sequentially pass through space 104′, separated by linkers 335, such labels respectively may cause changes in impedance that are detected by detection circuitry 160. Note that the labels within surrogate polymer 380 may be coupled to one another via linkers 335, and such linkers themselves may cause detectable changes in the impedance, and therefore resonant frequency of the tank circuit, such changes being different than, and distinguishable from, changes respectively caused by labels 131, 132, 133, 134. Further details regarding surrogate polymers are provided elsewhere herein. Illustratively, further details regarding XPANDOMERs™, their preparation, and their use in Sequencing By expansion (SBX™) are provided elsewhere herein.

In one nonlimiting example, the space between electrodes 102 and 103 is on the order of 10 nm (e.g., about 5-100 nm, or about 10-50 nm). Each unit of surrogate polymer may be configured to include two portions: (i) a label that causes a detectable change by the circuit, and (ii) a linker section 335 that causes another change, or no change at all, from baseline. The detectable label may be any suitable length, e.g., a length equivalent to about 10-20 nucleotides, or equivalent to about 20-50 nucleotides, such that only a single label 131, 132, 133, or 134 may reside within the space within the electrodes at any given time. The linker then separates adjacent signals. Accordingly, using surrogate polymers together with RF tank circuits in a manner such as provided herein whereby translocation is rapid, the method may provide a sequencing platform with high throughput and high accuracy. In particular, such an example may obviate the need to slow the free rate of translocation of the surrogate polymer strands through the nanopore, so nucleotides may be sequenced at rates exceeding about 1000 nt to 10,000 nt to 100,000 nt to 1,000,000 nt per seconds per nanopore.

It will be appreciated that fluid 120 described with reference to FIGS. 1A-1C may include any suitable combination of nucleotide analogues, ions, buffers, solvents, and the like. In some examples, fluid 120 may include at least one nucleotide analogue. Each of the nucleotide analogues may include a sugar, a nucleobase, a phosphate group, and a label. In some examples, the nucleobase (e.g., pyrimidine or purine) and phosphate group may be directly coupled to the sugar in a standard fashion, and the label may be indirectly coupled to the sugar via the phosphate group. For example, the nucleotides may have the structure:

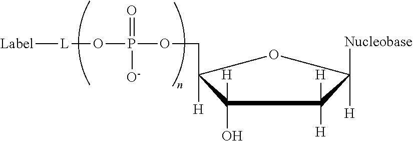

where n is greater than one (e.g., is 2, 3, 4, 5, 6, or greater than 6), and where L represents an optional linker coupling the label to the phosphate group. For example, the phosphate group may be selected from the group consisting of triphosphate, tetraphosphate, pentaphosphate, and hexaphosphate. Example linkers are described elsewhere herein. In other examples, the label may be coupled to the nucleobase. In other examples, the nucleotides may include xNTPs which are coupled between labels in a surrogate polymer (such as an Xpandomer) such as described elsewhere herein. In a manner such as described with reference to FIGS. 1A-1C, in various examples the labels may include polymers, beads or particles having different physical characteristics including permittivity, capacitance, inductance, magnetism, and/or ohmic resistances than one another that alter the impedance between electrodes 102, 103. Examples include metal particles (e.g., Au, Pt, Pd, Fe, or the like), magnetic particles, paramagnetic particles, beads (e.g., polystyrene, polypropylene, or the like), and polymers (e.g., PEG, protein, DNA, spermine, spermidine, or the like).

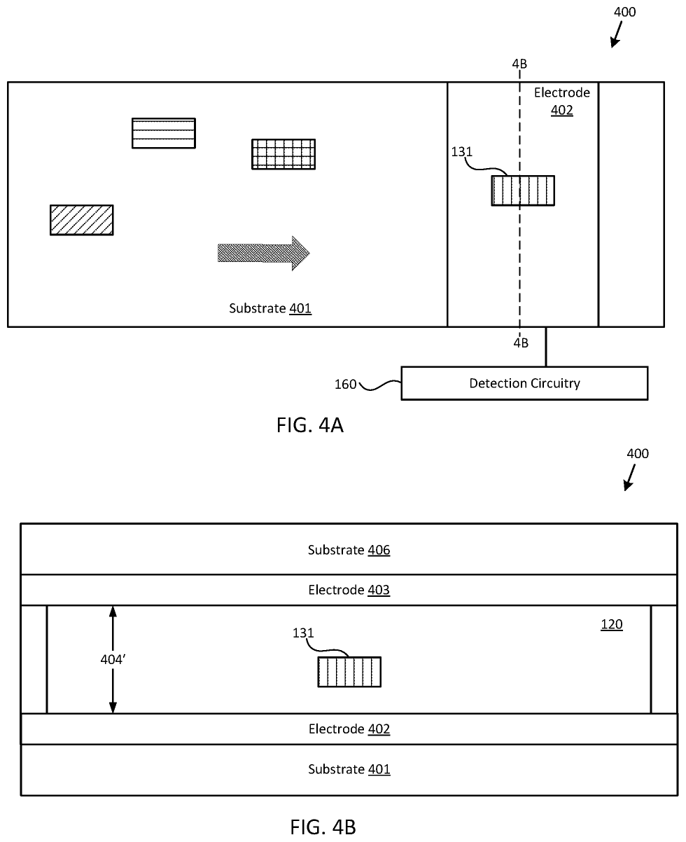

As noted further above, FIGS. 1A-1C and 3A-3D illustrate only one example of an electrode structure that may be used to detect changes in impedance caused by labels corresponding to nucleotides. For example, at least one of the electrodes instead may include a surface of a fluidic channel, such as a surface of a flowcell. Illustratively, FIGS. 4A-4B schematically illustrate alternative example compositions and systems for identifying nucleotides using changes in impedance between electrodes. System 400 illustrated in simplified plan view in FIG. 4A and in cross-section in FIG. 4B includes electrode 402 providing one surface of a fluidic channel, and electrode 403 providing another surface of the fluidic channel (electrode 403 omitted from FIG. 4A for ease of rendering). Electrode 402 may be disposed on substrate 401 and electrode 403 may be disposed on substrate 406. In a manner such as illustrated in FIG. 4A, electrodes 402 and 403 may be patterned so as to be disposed on only a portion of their respective substrate and thus define space 404′ through which fluid 120 flows between the electrodes. In a similar manner to that described with reference to FIGS. 1A-1C, 2A-2B, and 3A-3C, detection circuitry 160 may be coupled to electrodes 402 and 403 and configured to identify nucleotides using alterations in impedance that are caused by labels within fluid 120 (e.g., by label 131 illustrated in FIG. 4B). The labels may be coupled to the nucleotides when the labels pass between electrodes 402, 403, e.g., in a manner similar to that described with reference to FIGS. 1A-1C and 3A. Alternatively, the labels may be decoupled from the nucleotides when the labels pass between the electrodes, e.g., in a manner similar to that described with reference to FIGS. 3B-3D. In either circumstance, in some examples the labels may pass between the electrodes substantially one at a time, thus facilitating distinguishing between the changes in impedance caused by such labels. In other examples, multiple labels may pass between the electrodes at a given time, and the signals from those labels may be deconvolved from one another. For example, if K labels are simultaneously contributing to the detectable signal, there will be 4^{K} combinations of signals; where K equals 4, there are 256 combinations of signals which may be distinguished from one another.

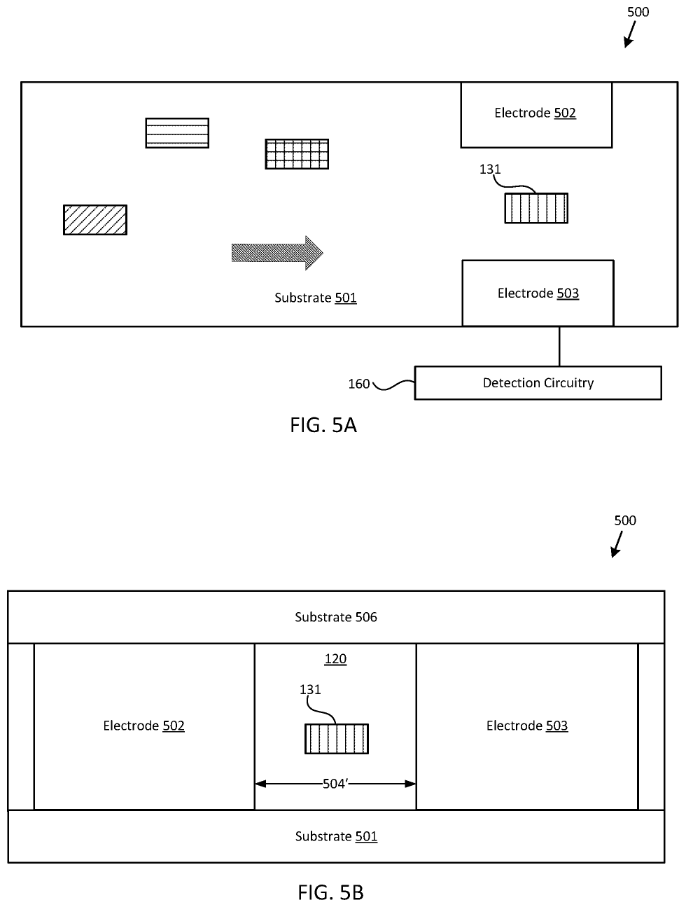

FIGS. 5A-5B schematically illustrate additional alternative example compositions and systems for identifying nucleotides using changes in impedance between electrodes. System 500 illustrated in simplified plan view in FIG. 5A and in cross-section in FIG. 5B includes electrode 502 providing one surface of a fluidic channel, and electrode 503 providing another surface of the fluidic channel. Electrodes 502 and 503 may be disposed between substrates 501 and 506. In a manner such as illustrated in FIG. 5A, electrodes 502 and 503 may be patterned so as to be disposed between only portion of the substrates and thus define space 504′ through which fluid 120 flows between the electrodes. In a similar manner to that described with reference to FIGS. 1A-1C, 2A-2B, and 3A-3C, detection circuitry 160 may be coupled to electrodes 502 and 503 and configured to identify nucleotides using alterations in impedance that are caused by labels within fluid 120 (e.g., by label 131 illustrated in FIG. 5B). The labels may be coupled to the nucleotides when the labels pass between electrodes 502, 503, e.g., in a manner similar to that described with reference to FIGS. 1A-1C, 3A, and 3D. Alternatively, the labels may be decoupled from the nucleotides when the labels pass between the electrodes, e.g., in a manner similar to that described with reference to FIGS. 3B-3C.

Note that examples such as described with reference to FIGS. 4A-4B and 5A-5B may include electrodes 106 and 107 (not specifically illustrated) across which detection circuitry 160 may apply a bias voltage to cause fluid to flow through the channels. For example, electrode 106 may be located near an inlet of the channel, and electrode 107 may be located near an outlet of the channel. In further instances, one or more additional electrodes may be included to apply a bias voltage to cause fluid to flow through the channels. In various examples, the bias voltage may be a DC bias voltage. In further examples, the bias voltage may vary so as to control characteristics of the fluid flow.

Optionally, in examples including a fluidic channel, multiple sets of electrodes may be provided along the channel to obtain multiple reads of the same label. Additionally, or alternatively, multiple fluidic channels may be aligned in parallel. Structures may be included that facilitate the unraveling of DNA as the DNA enters the channel (e.g., pillars such as known in the art). In some examples, the width of the channel(s) is less than the persistence length of DNA (which is about 50 nm), so that the DNA substantially may not form kinks or the like, and the possibility of co-translocation of multiple strands is decreased. Additionally, or alternatively, the inlets of the channels may share a common fluidic chamber and/or the outlets of the channels may share a common fluidic chamber. Additionally, or alternatively, the channels may share common electrodes 106 and 107 across which detection circuitry 160 may apply a bias voltage to cause fluid to flow through the channels, with each channel having its own set(s) of detection electrodes 402 and 403 or 502 and 503. In either example, the fluidic chamber at the inlet of the channel(s) (the same side as electrode 106) may include a library of polynucleotides, such as DNA, to sequence.

FIG. 8 illustrates an alternative configuration in which a biological pore 810 resides in a membrane 812 interposed between electrodes 102, 103 attached to the sides of a solid state aperture 814 which serves to hold the membrane 812. With a hybrid pore, the solid state structure's diameter opening is similar to the pore width (so no membrane is necessarily required), whereas in the example of FIG. 8, the diameter of the solid state structure 814 may be larger and a membrane 812 used to anchor the pore to the aperture 814. In various examples, the biological or hybrid pore may be fully or partially located between electrodes 102, 103, and such structure may reduce or inhibit the polynucleotide from forming one or more secondary structures (e.g., balling up) during translocation through the pore. Optionally, aperture 814 may be divided such that respective portions of it may function as electrodes 102 and 103.

Electrodes such as described with reference to FIGS. 1A-1C, 3A-3D, 4A-4B, 5A-5B, and 8 may have any suitable size, shape, and spacing to facilitate detecting change in impedance caused by labels 131, 132, 133, 134. Illustratively, the electrodes may be spaced apart from one another by about 10 nm to about 100 μm, or by about 100 nm to about 50 μm, or by about 1 μm to about 20 μm, or by 1 μm or less, or by about 100 nm or less, or about 10 nm or less. The electrode tips may have any suitable size, e.g., may be about 1 nm to about 1 μm wide, or about 1 nm to about 100 nm wide, or about 1 nm to about 50 nm wide, or about 1 nm to about 20 nm wide, or about 1 nm to about 10 nm wide, or about 5 nm to about 10 nm wide. It will be appreciated that regardless of the particular electrode configuration that is used, the labels may pass between the electrodes substantially one at a time, thus facilitating distinguishing between the changes in impedance caused by such labels. The labels may pass between the electrodes responsive to any suitable force, including but not limited to fluidic pressure, osmotic pressure, or application of a bias using electrodes 106 and 107 in a manner such as described elsewhere herein.

Detection circuitry 160 described with reference to FIGS. 1A-1C, 2A-2B, 3A-3D, 4A-4B, 5A-5B, and 8 may be configured to detect changes in impedance caused by labels that pass at any suitable rate between the electrodes. For example, the labels may pass between the electrodes at a rate of at least about one thousand per second, or at least about 10,000 per second, or at least about 100,000 per second, or even at a rate of at least about 1 million per second, and detection circuitry 160 may include electrical circuitry configured to measure the resulting changes in impedance in any suitable manner, e.g., by measuring a corresponding change in the resonant frequency of a tank circuit.

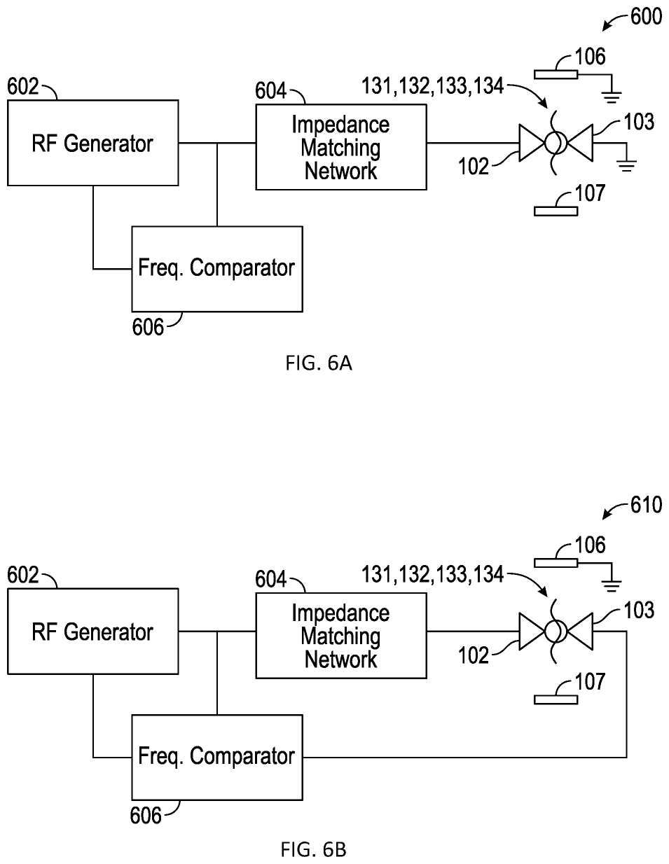

FIG. 6A illustrates an example tank circuit 600 that includes an RF generator 602 to generate an RF excitation signal to excite the electrodes 102, 103, an impedance matching network 604 to connect an output of the RF generator 602 to the electrodes 102, 103 so that the RF excitation signal is coupled to the electrodes 102, 103, and a frequency comparator 606 to compare (i) a frequency of reflected RF energy reflected from the electrodes 102, 103 to (ii) the frequency of the RF excitation signal. Additional electrodes 106 and 107 may be provided across which an applied bias voltage may cause fluid to flow through the space between electrodes 102, 103.

In various examples, the RF generator 602 may include a local oscillator. A local oscillator may generate the RF excitation signal having a first carrier frequency. The local oscillator may be a variable frequency oscillator controllable by a controller so that the first carrier frequency may be changed to accommodate detection of various different types of elements at various different rates. In further instances, the local oscillator may be a fixed oscillator.

The impedance matching network 604 may comprise a combination of inductors, capacitors, and resistors arranged to match an output impedance of the RF generator 602 to a nominal input impedance of the electrodes 102, 103. For instance, electrodes 102, 103 may have a nominal input impedance when elements are not passing through a space between the electrodes. In further examples, the impedance matching network establishes an intentional known impedance mismatch between the RF generator 602 and the electrodes 102, 103. An intentional known impedance mismatch may be implemented to obtain a reflected power of known quiescent magnitude. Such reflected power may be desired to be generated to facilitate measurement accuracy, circuit stability, and/or other operative capabilities as desired. In various examples, the impedance matching network 604 is connected at an input node to the RF generator 602, and connected at an output node to the electrode 102. Moreover, the impedance matching network 604 may have tunable components. For instance, one or more variable capacitor and/or variable inductor and/or variable resistor may be implemented. The value of these variable components may be varied to accommodate detection of various different types of elements at various different rates. In various examples, the tunable components are tuned by a controller in concert with changes to the first carrier frequency.

The frequency comparator 606 may also be connected to the input node of the impedance matching network 604. The frequency comparator 606 comprises a circuit to receive the reflected RF energy that is reflected from the electrodes 102, 103 through the impedance matching network 604 and back toward the RF generator 602. This reflected RF energy may be shunted at least partially from toward the RF generator 602 and to toward the frequency comparator 606. For instance, a circulator or other component may be implemented to shunt reflected RF energy at least partially toward the frequency comparator 606 while permitting the RF excitation signal to pass into the input node of the impedance matching network 604. The frequency comparator 606 is operable to compare a frequency of the reflected RF energy to a frequency of the RF excitation signal. A difference between the frequency of the reflected RF energy and the frequency of the RF excitation signal corresponds to a change in resonant characteristics of the tank circuit. For instance, an element passing through the gap between electrodes 102, 103 may change a resonant frequency of the tank circuit so that the reflected RF energy exhibits a different frequency than the RF excitation signal.

FIG. 6B illustrates an example tank circuit 610 that includes components similar to the aforementioned tank circuit 600 (FIG. 6A), with different interconnections. For instance, the RF generator 602 may generate an RF excitation signal to excite the electrodes 102, 103. A bias voltage applied to electrodes 106 and 107 may cause fluid to flow through the space between electrodes 102, 103.

The impedance matching network 604 may couple the output of the RF generator 602 to the electrodes 102, 103 so that the RF excitation signal is coupled to the electrodes 102, 103. However, the frequency comparator 606 may be connected differently in order to compare a frequency of transmitted RF energy coupled through the electrodes 102, 103 across the space between the electrodes to the frequency of the RF excitation signal. This is in contrast to the circuit of FIG. 6A, wherein the frequency of the reflected RF energy is compared to the frequency of the RF excitation signal.

The frequency comparator 606 may be connected to electrode 103 while the impedance matching network 604 may be connected to electrode 102. The frequency comparator 606 may also be connected to the RF generator 602. The frequency comparator 606 may compare (i) a frequency of transmitted RF energy that is passed across the space between the electrodes 102, 103 to (ii) the frequency of the RF excitation signal. The frequency of the transmitted RF energy may change in response to changes in the impedance of the tank circuit associated with the different elements passing through the space between the electrodes.

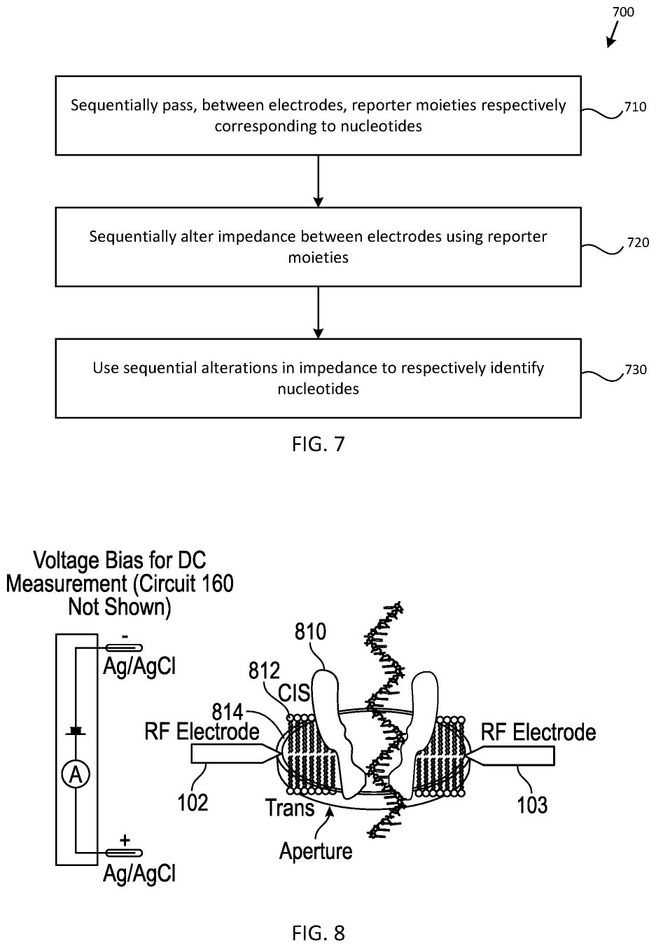

It will be appreciated that systems, compositions, and operations such as described with reference to FIGS. 1A-1C, 2A-2B, 3A-3D, 4A-4B, 5A-5B, 6A-6B, and 8 suitably may be adapted for use in various methods of polynucleotide sequencing, including but not limited to SBS or SBX, but may be used in any suitable application or context for which it is desirable to identify nucleotides. For example, FIG. 7 illustrates a flow of operations in an example method 700 for identifying nucleotides using changes in impedance between electrodes. Method 700 may include sequentially passing, through a space between electrodes, labels respectively corresponding to the nucleotides (operation 710). Example labels, example electrode structures, and example operations for passing labels through spaces between electrodes, are provided with reference to FIGS. 1A-1C, 3A-3D, 4A-4B, 5A-5B, and 8. Method 700 also may include sequentially altering an impedance between the electrodes using the labels (operation 720). Example manners in which the impedance between electrodes may be altered using labels are described with reference to FIGS. 1A-1C, 2A-2B, 3A-3D, 4A-4B, 5A-5B, and 8. Method 700 also may include using the sequential alterations in the impedance to respectively identify the nucleotides (operation 730). Example manners in which sequential alterations in impedance are described with reference to FIGS. 1A-1C, 2A-2B, 3A-3D, 4A-4B, 5A-5B, 6A-6B, and 8.

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of identifying nucleotides, the method comprising:
sequentially passing, through a space between electrodes, labels respectively corresponding to the nucleotides;
sequentially altering an impedance between the electrodes using the labels; and
using the sequential alterations in the impedance to respectively identify the nucleotides.

2. The method of claim 1, wherein:
the electrodes are part of a resonating circuit that has a resonant frequency based upon the impedance between the electrodes;
the sequential alterations in the impedance respectively cause changes in the resonant frequency; and
the nucleotides are identified using the respective changes in the resonant frequency.

3. The method of claim 2, wherein the resonating circuit comprises a tank circuit.

4. The method of claim 1, wherein the labels change the impedance by altering the capacitance within the resonating circuit that causes the changes in the resonant frequency.

5. The method of claim 1, wherein the sequential alterations in the impedance correspond to a sequence in which a polymerase adds the nucleotides to a first polynucleotide using a sequence of a second polynucleotide.

6. The method of claim 1, wherein the labels pass through the space between the electrodes while the labels are coupled to the nucleotides.

7. The method of claim 1, wherein the labels pass through the space between the electrodes after the labels are cleaved from the nucleotides.

8. The method of claim 1, wherein the labels pass through the space between the electrodes after the nucleotides are cleaved from a polynucleotide.

9. The method of claim 1, wherein the labels are coupled to one another in a surrogate polymer passed through the space between the electrodes.

10. The method of claim 1, wherein the labels are coupled to respective gamma phosphates of the nucleotides.

11. The method of claim 1, wherein the space between the electrodes comprises a nanopore.

12. (canceled)

13. The method of claim 11, wherein at least one of the electrodes comprises a respective portion of the nanopore.

14. The method of claim 1, wherein at least one of the electrodes comprises a surface of a fluidic channel.

15. (canceled)

16. The method of claim 1, wherein the electrodes are spaced apart from one another by about 100 nm or less.

17. The method of claim 1, wherein tips of the electrodes have widths of about 1 nm to about 100 nm.

18. The method of claim 1, wherein the labels pass through the space between the electrodes responsive to application of a voltage.

19. The method of claim 1, wherein the labels pass through the space between the electrodes at a rate of at least one thousand per second.

20. (canceled)

21. The method of claim 1, wherein the labels comprise polymers, beads, or particles having different physical characteristics than one another that alter the impedance between the electrodes.

22. The method of claim 21, wherein the different physical characteristics are selected from the group consisting of permittivity, capacitance, inductance, magnetism, and ohmic resistance.

23-25. (canceled)

26. A system for identifying nucleotides, the system comprising:
electrodes spaced apart from one another,
labels corresponding to the nucleotides, the labels being for passing through the space between the electrodes and for altering an impedance between the electrodes; and
circuitry coupled to the electrodes and configured to identify the nucleotides using the alterations in the impedance.

27-50. (canceled)
